(19) United States
(12) Patent Application Publication
(10) Pub. No.: US 2020/0218096 A1
(43) Pub. Date: Jul. 9, 2020

(54) APPARATUS AND METHOD FOR FITTING HEAD MOUNTED VISION AUGMENTATION SYSTEMS

(71) Applicant: ESIGHT CORP., Kanata (CA)

(72) Inventors: ROBERT HILKES, OTTAWA (CA); FRANK JONES, CARP (CA); KEVIN RANKIN, OTTAWA (CA)

(21) Appl. No.: 16/822,731

(22) Filed: Mar. 18, 2020

Related U.S. Application Data
(63) Continuation of application No. 15/585,809, filed on May 3, 2017.
Application No. 15/585,809 is a continuation of application No. 14/758,623, filed on Jun. 30, 2015.
(60) Provisional application No. 61/747,380, filed on Dec. 31, 2012.

(51) Int. Cl. G02C 13/00; G02C 5/12; G02C 3/02; G02C 11/00; A61B 3/04; G02B 27/01

(52) U.S. Cl. CPC G02C 13/005; G02C 5/124; G02C 3/02; G02C 11/10; G02C 5/126; G02B 2027/0156; G02B 27/0176; G02B 2027/0178; G02C 2200/02; G02B 2027/0158; A61B 3/04

(57) ABSTRACT

A head worn display can be designed with an integrated camera for obtaining an image of a scene, transmitting the obtained image to a processor, modification of the image in substantially real time by the processor, and displaying the modified image on a display device worn by the individual. According to embodiments of the invention various methods are provided for adjusting the position of the displayed video in the horizontal left/right, vertical up/down, and horizontal in/out, and angular up/down dimensions relative to the individual's eyes.

### CROSS-REFERENCE TO RELATED APPLICATIONS

This patent application claims the benefit of priority as a continuation of U.S. patent application Ser. No. 15/585,809 filed on May 3, 2017 entitled “Apparatus and Method for Fitting Head Mounted Vision Augmentation Systems,” which itself claims the benefit of priority as a continuation of U.S. patent application Ser. No. 14/758,623 filed on Jun. 30, 2015 entitled “Apparatus and Method for Fitting Head Mounted Vision Augmentation Systems,” which itself claims the benefit of priority from World Patent Application PCT/CA2013/001,077 filed on Dec. 30, 2013 entitled “Apparatus and Method for Fitting Head Mounted Vision Augmentation Systems,” which itself claims the benefit of priority from U.S. Provisional Patent Application 61/747,380 filed on Dec. 31, 2012 entitled “Apparatus and Method for Fitting Head Mounted Vision Augmentation Systems,” the entire contents of these being incorporated herein by reference.

### FIELD OF THE INVENTION

The invention relates to head mounted vision augmentation displays and more specifically to fitting and adjusting such head mounted vision augmentation displays for improved performance and comfort.

### BACKGROUND OF THE INVENTION

The use of head mounted or spectacle-mounted video display systems for vision augmentation of users is becoming more prevalent both for addressing visual impairments and augmenting reality. Augmenting reality applications can include, but are not limited to, medicine, visual assistance, engineering, aviation, tactical, gaming, sports, virtual reality, environment simulation, and data display.

The inventors have invented a head-worn display (HWD) system, also referred to as a head mounted display (HMD), which may derive its image source from a video camera mounted similarly for the user. The HWD may also display the image from the video camera after applying one or more processing algorithms/effects to address visual impairments of the user as well as combining the raw/processed source image with content derived from a variety of electronic sources such as local and/or remote multimedia content. In other scenarios non-camera based content may be displayed alone. Such systems are particularly beneficial to, and have been designed specifically for, people who are visually impaired, that is, those affected by one of many possible diseases or eye conditions which reduce their functional visual performance relative to that of a normally sighted person. Such impairments may occur in one or both eyes and be present in different combinations in different individuals.

An essential aspect for such systems is the means by which the video display is aligned with the individual's physiology; namely the location and spacing of their eyes, the height of their nose and so forth. This aspect will determine in many instances the wearer's visual fatigue and/or stress of accommodating the images either discretely or overlaid with their normal field of vision. Furthermore, since HWDs by virtue of the one or more electronic video display(s), additional optical components, and potentially camera are typically heavier and bulkier than traditional spectacle eyeglasses, the physical design characteristics of the system need to be such that the wearer's comfort is maximized. Whilst in some applications, e.g. augmented reality, the user may wear the HWD for short periods of time in those addressing visual impairments the user may need to wear the HWD during all of their routine daily activities and hence for extended periods of time.

Amongst the significant challenges in optimizing the visual experience for wearers of HWDs is establishing the appropriate alignment of the display optics with the user's pupil(s). This challenge is further complicated when the user also wears prescription eyeglasses, as the head worn display and the user's eyeglasses often interfere with one another, detracting from the users visual experience, and causing physical discomfort. Within the prior art HWDs have typically been considered as the only optical element in front of the user's eyes and in many prior art HWDs the external world is blocked out. Accordingly, it would be a beneficial feature of a HWD therefore, to provide the ability to adjust the horizontal (left/right), vertical (up/down), horizontal (in/out), and angular (up/down) position of the display optics and the user's prescription ophthalmic lens(es) relative to their pupil(s). This user dependent configuration becomes even more complicated when considering the requirement to perform this both eyes of the wearer wherein two sets of displays optics need to be aligned with two corrective ophthalmic lenses with different prescriptions and all of this within a single easy to use assembly for the wearer with the potential for low cost visual augmentation and augmented reality applications.

It would also be beneficial, as with a user's visit to an optometrist for checking/adjusting their ophthalmic prescription, to provide these useful features within an HWD with the ability to determine all these necessary fitting and alignment parameters in a clinical setting, such as an ophthalmologist or optometrist, enabling the user to try the HWD in conjunction with their prescription lenses thus improving the fit for their visual prescription and performance, and their anthropometric dimensions. The fitting and optical prescription parameters thus established for the HWD could then be readily used to assemble a fully customized, user specific version of the HWD.

Other aspects and features of the present invention will become apparent to those ordinarily skilled in the art upon review of the following description of specific embodiments of the invention in conjunction with the accompanying figures.

### SUMMARY OF THE INVENTION

It is an objective of the present invention to mitigate drawbacks in the prior art in improving the comfort and optical alignment of head worn displays for users.

In accordance with an embodiment of the invention there is provided a device comprising:
(i) an ophthalmic assembly to be worn by a user having a visual defect allowing the mounting of an electronic assembly;
(ii) the electronic assembly comprising at least a camera for obtaining an image of a scene viewed by the user and an electronic processor for receiving image data from the camera and modifying the image data substantially in real time in dependence upon at least a characteristic of the user's visual defect to generate modified image data for display to the user via a near-to-eye display also forming part of the electronic assembly; wherein
   the ophthalmic assembly may be configured to accommodate a prescription lens to a prescription for the user and electronics assembly may be configured to appropriately position the near-to-eye display in the appropriate position relative to the user's eye.

In accordance with an embodiment of the invention there is provided various methods for calibrating the horizontal (left/right), vertical (up/down), horizontal (in/out), and angular (up/down) position of said head worn display relative to the user's eyes.

An ophthalmic assembly to be worn by a user having a visual defect allowing for:

(a) the demountable attachment of an electronic assembly comprising at least a camera for obtaining an image of a scene viewed by the user and an electronic processor for receiving image data from the camera and modifying the image data substantially in real time in dependence upon at least a characteristic of the user's visual defect to generate modified image data for display to the user via a near-to-eye display also forming part of the electronic assembly;
(b) the mounting of a prescription lens to a prescription for the user within the ophthalmic assembly;
(c) establishment of refinements in a prescription for the user when using the electronics assembly and near-to-eye display in conjunction with their normal vision;
(d) the mounting of at least a trial lens of a plurality of trial lenses during configuration of the ophthalmic assembly and electronics assembly to be configured to appropriately position the near-to-eye display in the appropriate position relative to the user's eye.

In accordance with an embodiment of the invention there is provided an electronic assembly comprising at least a camera for obtaining an image of a scene viewed by the user and an electronic processor for receiving image data from the camera and modifying the image data substantially in real time in dependence upon at least a characteristic of the user's visual defect to generate modified image data for display to the user via a near-to-eye display also forming part of the electronic assembly, wherein the electronic assembly may be demountably attached to an ophthalmic assembly configured to accommodate a prescription lens to a prescription for the user and electronics assembly may be configured to appropriately position the dear-to-eye display in the appropriate position relative to the user's eye.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures, wherein:

FIG. 1 provides a reference frame for the three dimensions “X”, “Y”, and “Z” that are used to describe the relationship of the head worn display and camera system relative to the wearer;

FIG. 2 depicts in more detail, the relationship between the camera and display optics in the HWD and prescription lenses, relative to the wearer's eye wherein the fitted head worn display is aligned such that the exit pupil of the display system, the optical center of the wearer's refractive prescription lenses, and the wearer's pupil, are all optically aligned;

FIG. 3 depicts a headband affixed to the temple arms of the glasses, and in firm contact with the wearer's forehead, according to an embodiment of the invention;

FIG. 4 depicts an adjustment method for affixing a headband to the temple arms of the glasses according to an embodiment of the invention;

FIG. 5 depicts a method of attaching an inner Ophthalmic Assembly 10 to an outer Electronics Assembly 2 of a head worn display according to an embodiment of the invention;

FIGS. 6A through 6C depict the orientation of a head worn display relative to the wearer's horizontal angle of sight for a head worn display according to an embodiment of the invention;

FIG. 7 depicts an ophthalmic frame incorporating temple arms, and ophthalmic prescription lenses according to an embodiment of the invention wherein the inner ophthalmic frame can be affixed to the outer electronic assembly through various means;

FIGS. 8A and 8B depict a region of video image selected from a much larger video image, based on commands from the wearer;

FIGS. 9 and 10 depict, for clarity, further embodiments of the invention;

FIG. 11 depicts an embodiment of the invention wherein a Rigid Mounting Rail 15 upon which the left and right HWD Display Optics 4 can travel in the “X” direction in order to accommodate different pupil spacings;

FIG. 12 depicts an embodiment of the invention in which a Display Optics Position Clamp 17 is held in place with two clamp screws 18, firmly mating with the Clamp Surface 19 on the HWD Display Optics 4, thereby preventing said HWD Display Optics 4 from travelling in the left/right “X” dimension once in place;

FIG. 13 depicts vertical height adjustment in the “Y” direction, of the Node Bridge Assembly 16 in the Ophthalmic Assembly 10, for a head-worn display according to an embodiment of the invention;

FIG. 14 depicts the in/out horizontal adjustment in the “Z” direction, of the Node Bridge Assembly 16, achieved through providing Nose Bridge Assemblies 16 of varying dimensions according to an embodiment of the invention;

FIG. 15 depicts another view of the Ophthalmic Assembly 10, showing in particular, the Magnetic Bioptic Hinge (Male Portion) 8B according to an embodiment of the invention;

FIG. 16 depicts another view of the Electronics Assembly 2, showing in particular, the Magnetic Bioptic Hinge (Female Portion) 8A according to an embodiment of the invention;

FIG. 17 shows the Electronics Assembly 2, with the Display Optics Position Clamp 17 removed, and the Clamp Surface 19 on the HWD Display Optics Assembly 4 according to an embodiment of the invention;

FIG. 18 depicts a head-worn or spectacle mounted video display system according to an embodiment of the invention and its connectivity to ancillary processing and control electronics; and

FIG. 19 depicts schematically the electronic elements of head-worn or spectacle mounted video display system and ancillary electronic devices according to an embodiment of the invention.

### DETAILED DESCRIPTION

The present invention is directed to head worn displays and more specifically to augmenting sight for people with vision loss.

The ensuing description provides exemplary embodiment(s) only, and is not intended to limit the scope, applicability or configuration of the disclosure. Rather, the ensuing description of the exemplary embodiment(s) will provide those skilled in the art with an enabling description for implementing an exemplary embodiment. It being understood that various changes may be made in the function and arrangement of elements without departing from the spirit and scope as set forth in the appended claims.

A “head worn display” (HWD) as used herein and throughout this disclosure refers to a wearable electronic device that incorporates an image capturing device and an image presentation device operating in conjunction with a microprocessor such that an image captured by the image capturing device can be modified in substantially real time and subsequently presented to the user on the image presentation device. Alternatively in some cases, the source of the image for display to the wearer of the HWD may come from a remotely attached camera or any video source. The microprocessor and any associated electronics including, but not limited to, memory, user input device, context determination, graphics processor, and multimedia content generator may be integrated for example with the HWD, form part of an overall assembly with the HWD, or as discrete unit connected either wirelessly or with a wire to the HWD. Said microprocessor could also be a personal electronic device such as a smart phone, connected either with a wire or wirelessly to the HWD.

A “wearer”, “user” or “patient” as used herein and through this disclosure refers to, but is not limited to, a person or individual who uses the HWD either as a patient requiring visual augmentation to fully or partially overcome a vision defect or as an ophthalmologist, optometrist, optician, or other vision care professional preparing a HWD for use by a patient. A “vision defect” as used herein may refer to, but is not limited, a physical defect within one or more elements of a user's eye, a defect within the optic nerve of a user's eye, a defect within the nervous system of the user, a higher order brain processing function of the user's eye, and an ocular reflex of the user.

It is conceivable to one skilled in the art, that a “wearer” or “user” could also be an individual with healthy vision, using the HWD in an application other than for the purposes of ameliorating physical vision defects. Said applications could include, but are not necessarily limited to gaming, augmented reality, night vision, thermal imaging, computer use, viewing movies, environment simulation, etc. Augmented reality applications may include, but are not limited to, medicine, visual assistance, engineering, aviation, tactical, gaming, sports, virtual reality, environment simulation, and data display.

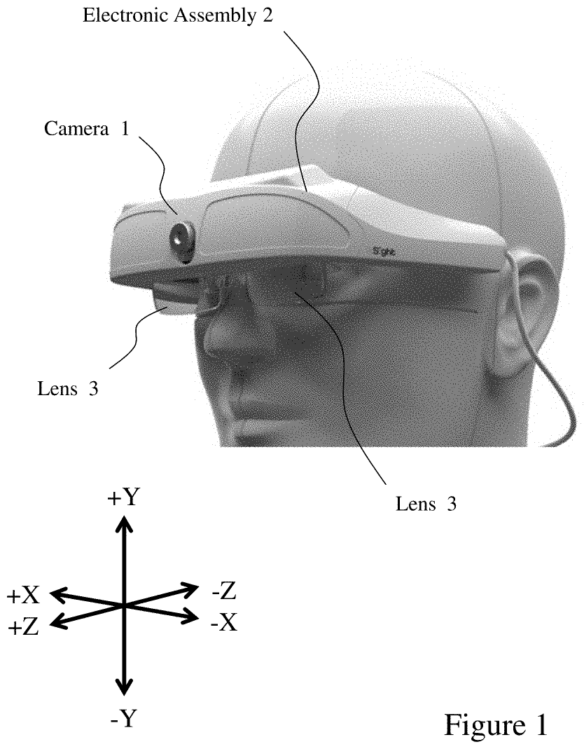

FIG. 1 provides a reference frame for the three dimensions “X”, “Y”, and “Z” that are used to describe the relationship of the head worn display, comprising an Electronics Assembly 2 and Camera 1, and Lenses 3 relative to the wearer. The “X” dimension as shown indicates the position of the head worn system laterally across the left right dimension of the wearer's face. Generally, the “X” dimension values increase in a rightward direction relative to the wearer's perspective, and decreases in a leftward direction relative to the wearer's perspective. X=0 is considered to be the center of the user's nose. Similarly the “Y” dimension values increase in an upward direction and decrease in a downward direction whilst “Z” dimension values increase in the direction moving away from the wearer's face, and decrease in the direction moving closer to the wearer.

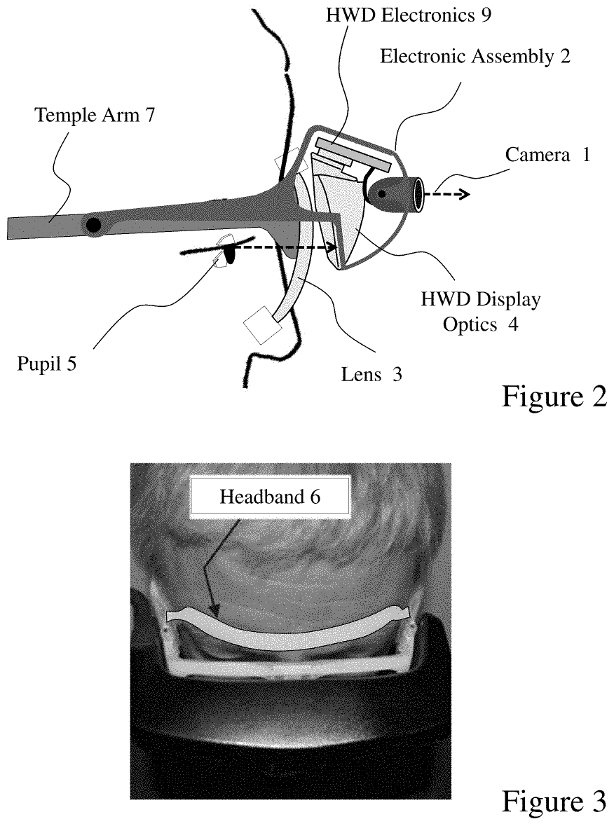

Referring then to the accompanying FIGS. 2 to 19, a HWD generally comprises an Ophthalmic Assembly 10 and an Electronic Assembly 2, said Ophthalmic Assembly 10 may comprise left and right temple arms 7, and a front structure joining the Temple Arms 7 which can optionally accommodate Lenses 3, and a nose bridge. Said Electronic Assembly 2 portion of the HWD itself comprises left and/or right HWD Display Optics 4 and HWD electronics 9 together with in many instances a Camera 1 or Cameras 1. In some augmented reality applications the HWD may overlay content from a source other than a Camera 1. In visual augmentation applications the Camera 1 typically captures the wearer's field of view which may be modified in dependence upon the wearer's visual dysfunction(s) prior to being presented to the wearer. Said Lenses 3 may according to embodiments of the invention be ophthalmic trial lenses, a plano lens with cross hairs for fitting adjustment purposes, ophthalmic lenses to the wearer's prescription, and sunglasses.

A head-worn display (HWD) or otherwise called head-mounted, or head-borne display, uses a near-to-eye, head-mounted or spectacle-mounted display, in which the screen is typically less than an inch in size, will also exploit special HWD Display Optics 4 which are designed to project the screen image onto the wearer's retina, discretely or in conjunction with the wearer's field of view, giving the wearer the perception of viewing a larger display at a distance. According to embodiments of the invention these left and right HWD Display Optics 4 project the image or images to the user through the individual's prescription lenses 3, or contact lenses, which may be employed with HWDs according to embodiments of the invention but are not explicitly described in all embodiments of the invention, which provide refractive correction wherein the display is used in conjunction with the individual's eyesight. In other embodiments of the invention the display(s) provide the sole optical input to the individual's eye or the display(s) provide additional optical input to the wearer without the wearer having corrective lenses. In other embodiments a single display is used with either the left or right eye whereas in others two displays are used, one for each eye, or a single display is used generating images for both the left eye and/or right eye.

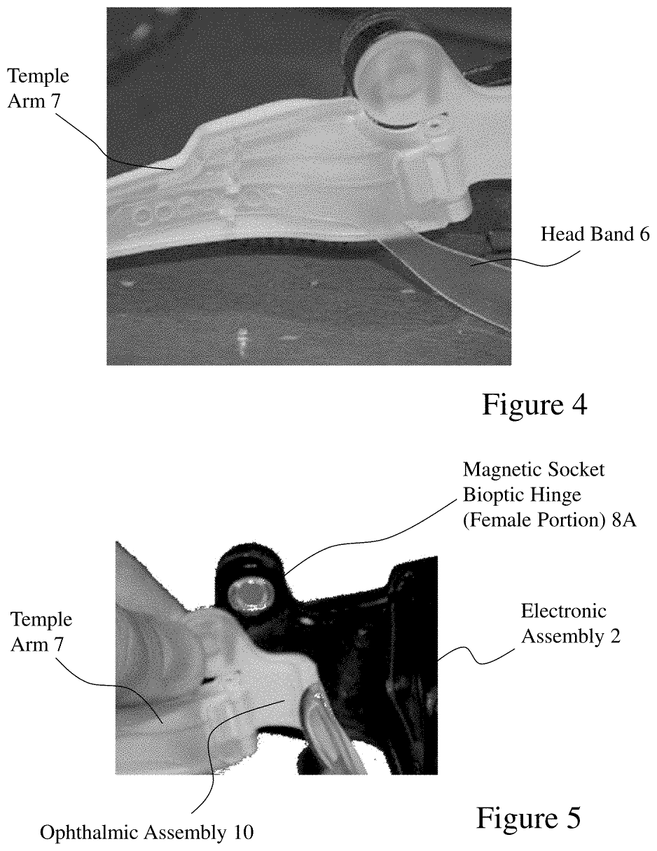

One of the significant challenges in developing head borne displays has been the precise alignment of the left and right HWD Display Optics 4 with the wearer's Pupil 5 as depicted in FIG. 2. HWDs are frequently large and heavy compared with normal eyeglasses, and as such, care must be taken to ensure the secure and proper alignment of the HWD Display Optics 4, while minimizing the physical discomfort of the wearer. Traditionally HWDs deployed various methods of tensionable or elastic head straps to ensure minimal relative movement between the HWD Display Optics 4 and the wearer's Pupils 5. Such tensionable or elastic head straps generally wrap round the back of the wearer's head. In contrast, as evident from FIGS. 3 and 4, an HWD according to an embodiment of the invention exploit a Headband 6 which attaches to the Temple Arms 7 in an adjustable manner such that the Headband 6 sits upon the wearer's forehead thereby removing loading to the user's nose where the HWD nose bridge fits as well as their ears via offsetting loading through the Temple Arms 7 and potentially cheeks if the HWD contacts them. As depicted in FIG. 4 the Headband 6 may comprise at either side a series of holes that fit a projection on each Temple Arm 7 so that the wearer can adjust the Headband 6 and maintain the set Headband 6 length established.

Now referring to FIGS. 3 and 4 there is depicted another aspect of the invention in respect of a headband 6, which spans the distance between the two Temple Arms 7 of the Ophthalmic Assembly 10, and whose length is adjusted such that it snugly touches the wearer's forehead. This headband 6 in contact with the wearer's forehead, used in conjunction with the Temple Arms 7, and a simple headstrap, not identified for clarity, around the back of the wearer's head as is commonly found with sports sunglasses for example, serves to create a snug load bearing “halo” around the wearer's head, which can hold the HWD Display Optics 4 and Ophthalmic Assembly 10 in firm fixed alignment with the wearer's Pupil 5, in spite of the wearer's head movements. This same aspect of the invention also serves the purpose of transferring some of the weight of the Electronic Assembly 2 from the wearer's nose, as imparted by the Node Bridge Assembly 16, onto the wearer's forehead, wherein the weight can be distributed across a much larger area.

Ideally, the HWD display optics is correctly aligned in three dimensions to the wearer's eyes. The X dimension as shown in FIG. 1 refers to the left/right alignment of the HWD Display Optics 4 relative to the wearer's Pupils 5. The Y dimension as shown in FIG. 1 refers to the up/down alignment relative to the wearer's Pupils 5. Finally, the Z dimension as shown in FIG. 1 refers to the in/out dimension of the HWD relative to the wearer's Pupils 5, or stated differently, how far the HWD Display Optics 4 are from the wearer's Pupils 5. Positive values or changes of these dimensions represent right, up, and out relative to the wearer whilst negative values or changes of these dimensions represent left, down, and in relative to the wearer.

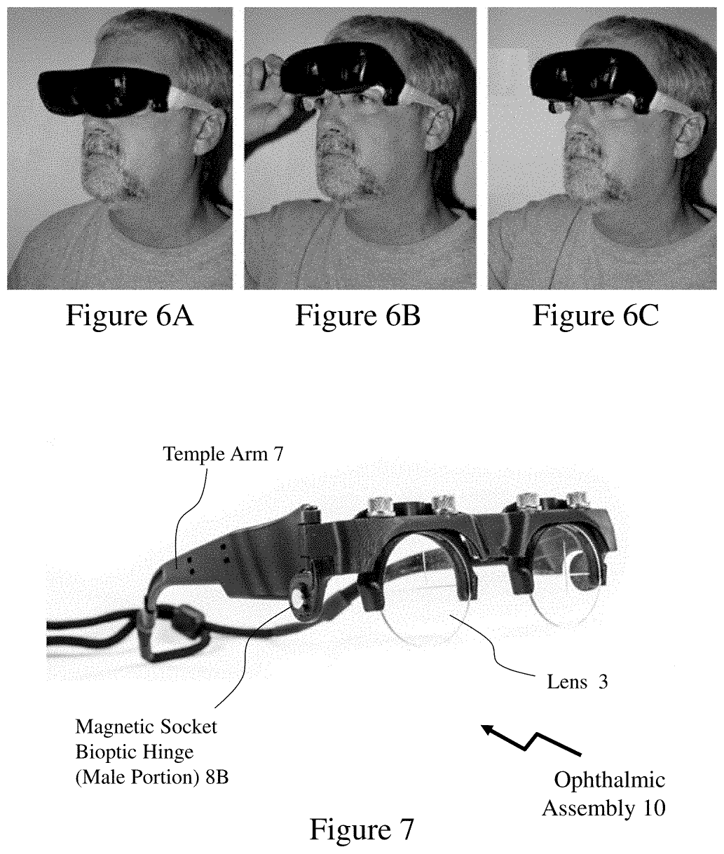

According to an embodiment of the invention, the Ophthalmic Assembly 10 can be hinged relative to the Electronic Assembly 2, such that the wearer can easily pivot the HWD Display Optics 4 up/down according to their preferences and the task they are engaged in, selecting whether they wish to view the HWD Display Optics 4 in a full time immersive posture, such as depicted in FIG. 6A or part time bioptic posture depicted in FIG. 6C. Said pivot point may be in the same axis as that for the natural rotation of the human eyeball, so that proper optical alignment exists at all times between the HWD Display Optics 4 and the wearer's Pupil 5. In other embodiments of the invention the transition between full immersive and partial bioptic postures may be based upon the wearer's head tilt, see for example US Patent Publication 2012/0,306,725 entitled “Apparatus and Method for a Bioptic Real Time Video System.”

Also according to an embodiment of the invention, the hinged axial relationship between the Ophthalmic Assembly 10 and the Electronic Assembly 2 may be detachable, such as through the use of magnetic couplings in a keyed channel or other variations as would be evident to one skilled in the art. Such magnets may for example include rare earth magnets such as samarium-cobalt and neodymium-iron-boron (NIB) for example as well as other permanent magnets including, but not limited to, “hard” ferromagnetic materials such as alnico and ferrite. In this manner, the Ophthalmic Assembly 10 can be easily detached from the Electronic Assembly 2 for the purposes of cleaning, such as depicted in FIG. 5 wherein in addition to the Ophthalmic Assembly 10 and the Electronic Assembly 2 there are identified the Temple Arm 7 and the Female Portion of the Magnetic Bioptic Hinge 8A. However, it would be evident to one skilled in the art that other mechanisms for providing detachable interconnection between the Ophthalmic Assembly 10 and Electronic Assembly 2 can be provided without departing from the scope of the invention. Some of these may require use of a tool to permit detachment whereas others may not.

Accordingly, as depicted in FIGS. 6A through 6C the orientation of a head worn display relative to the wearer's horizontal angle of sight for a head worn display according to an embodiment of the invention can be easily changed by the user. In FIG. 6A, the wearer is immersed such that the head worn display is horizontally aligned with a comfortable forward viewing eye angle. In FIG. 6B the wearer is adjusting the angle of the display relative to their head. In FIG. 6C the display is in a bioptic orientation, allowing the wearer to look beneath the video display by looking out at a normal angle, and up into the display by tilting the angle of their gaze upward.

An advantage of a magnet coupling to enable the mating and easy disconnection of the Ophthalmic Assembly 10 and the Electronic Assembly 2 is that different Ophthalmic Assemblies 10 can be easily and rapidly affixed and detached to a common Electronic Assembly 2. Accordingly, within an embodiment of the invention one can envision, for example, an Ophthalmic Assembly 10 that is designed to carry trial lenses such as are commonly used by ophthalmologists and optometrists when establishing a patients optical prescription. In this manner, a wearer could use the Electronic Assembly 2 on a trial basis for example, even though their personal lens prescription has not been incorporated into an Ophthalmic Assembly 10, by having a trained ophthalmologist or optometrist copy their lens prescription using trial lenses. Equally, adjustments to the wearer's prescription(s) may be evaluated given the combination of far-field natural visual elements with near-field display generated elements. Further, as will become evident in descriptions relating to other aspects of the fitting and customization of the Ophthalmic Assembly 10 and Electronics Assembly 2 other optical elements, such as plain glass with crosshairs/gridlines etc. may be employed to aid/enhance the processes.

Referring to FIG. 7 there is depicted an Ophthalmic Assembly 10 incorporating Temple Arms 7, and ophthalmic prescription lenses, Lenses 3, according to an embodiment of the invention wherein this inner ophthalmic frame, namely Ophthalmic Assembly 10, can be affixed to the outer electronic assembly, Electronic Assembly 2 which is not shown for clarity, via the magnetic attachment. Within FIG. 7 the Male Portion 8B of the Magnetic Bioptic Hinge is depicted on the sides of the Ophthalmic Assembly 10.

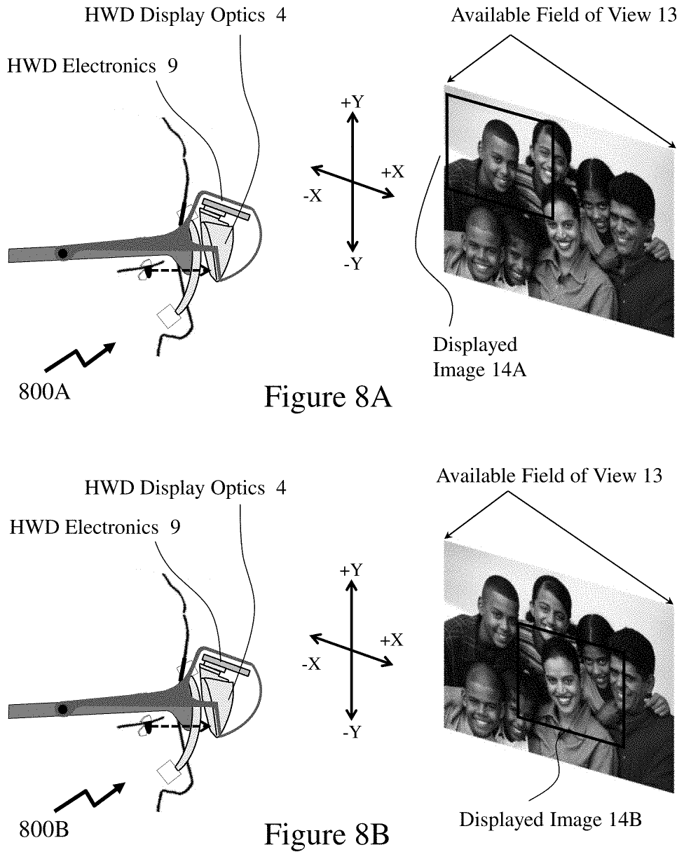

Referring to FIGS. 8A and 8B there are depicted first and second images 800A and 800B respectively wherein a user wearing a HWD comprising HWD Electronics 9 and HWD Display Optics 4 acquires an available field of view 13 and presents in each instance first and second Displayed Images 14A and 14B respectively representing the upper left and central mid points of the available field of view 13. Accordingly, it would be evident that wherein the first and second Displayed Images 14A and 14B respectively are presented to the wearer as their full vision then the selection of the Lenses 3 is determined primarily from best performance of coupling the display(s) to their Pupils 5. However, where the first and second Displayed Images 14A and 14B respectively are presented as an overlay to the user's normal vision of the available field of view 13 then the Lenses 3 may be balanced to the normal vision of the wearer, to present the available field of view 13 and the selected one of the first and second Displayed Images 14A and 14B respectively. Depending upon the balance of original field of view and displayed image then the final prescription employed for the Lenses 3 may be adjusted. Accordingly, finding the appropriate prescription may require the user to wear and use the HWD for extended periods of time to establish the correct balance between performance and fatigue/stress.

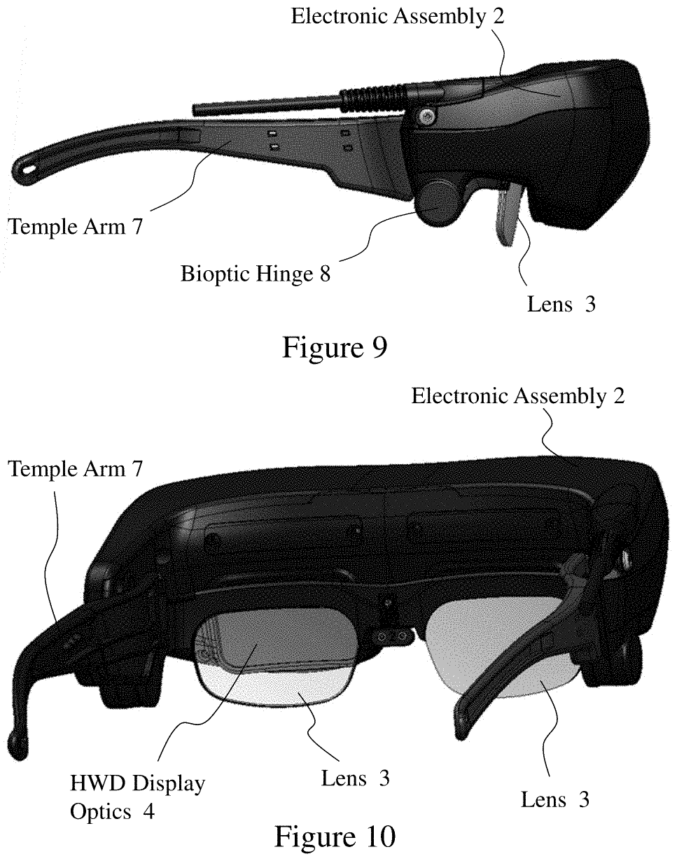

Referring to FIGS. 9 and 10 side and rear views respectively of a HWD according to an embodiment of the invention are presented showing the Electronic Assembly 2 in conjunction with the Lenses 3 and Temple Arms 7. In FIG. 9 the Bioptic Hinge 8 is evident on the side of the Electronic Assembly 2 whilst in FIG. 10 the HWD Display Optics 4 are evident through the Lens 3.

Such a trial lens frame as described supra in respect of FIG. 7 could also be used without the Electronics Assembly 2 attached, in order to confirm that the wearer's correct prescriptions lenses have been properly copied with trial/final lenses where the lenses for the HWD are of a different design to those in the wearer's standard prescription eyewear. Furthermore, said trial frame could be designed so that the trial lenses can be shifted in the +/−X direction, for proper horizontal alignment with the wearer's pupil.

Furthermore, the same Ophthalmic Assembly 10 could be used with plano trial lenses for example, said plano lenses being marked with vertical and horizontal crosshairs, which could be used to visually align the center of the trial lenses with the wearer's pupil. The cross haired trial lens could be keyed, for example, such that their angular position in the trial lens holder is fixed.

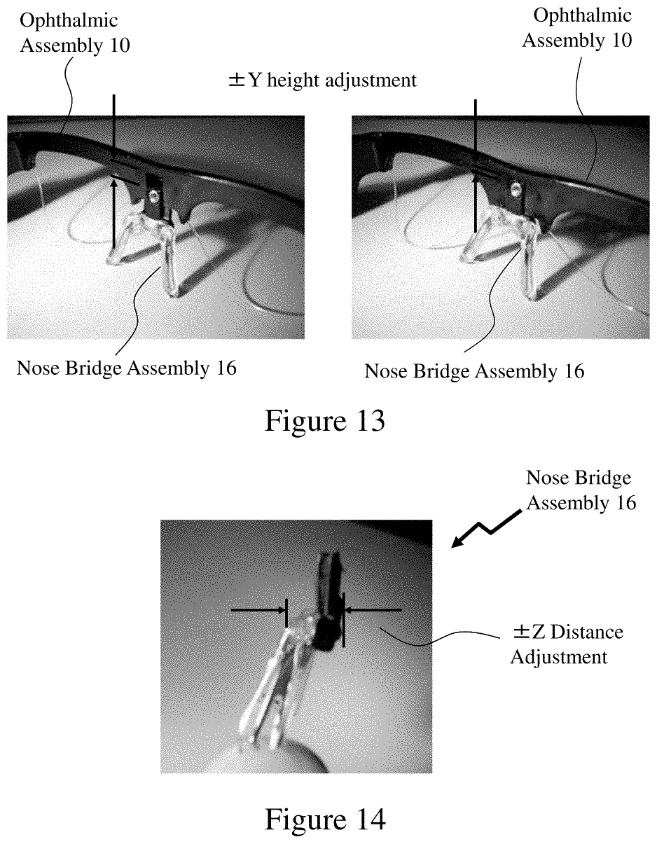

Furthermore, the same Ophthalmic Assembly 10 could be designed such that different depths of Node Bridge Assembly 16 could be affixed to the Ophthalmic Assembly 10, each designed to place the Ophthalmic Assembly 10 and the prescription lenses at a slightly different distance Z from the wearer's face. Finally, said Node Bridge Assembly 16 could be constructed such that its height can be varied relative to the Ophthalmic Assembly 10, by sliding its locations vertically up/down in a channel, and affixing the Node Bridge Assembly 16 in place using a simple set screw such as depicted in FIGS. 13 and 14.

Through all of the above described steps, a trained ophthalmologist or optometrist can confirm that the wearer's lens prescription has been properly copied and/or established, that their left and right pupil spacing has been properly set, that the distance of the HWD Display Optics 4 from the wearer's Pupil 5 has been properly set by selecting the appropriate Node Bridge Assembly 16, and lastly, the height of the prescription lenses 3 has been properly established by adjusting the vertical position of the Node Bridge Assembly 16.

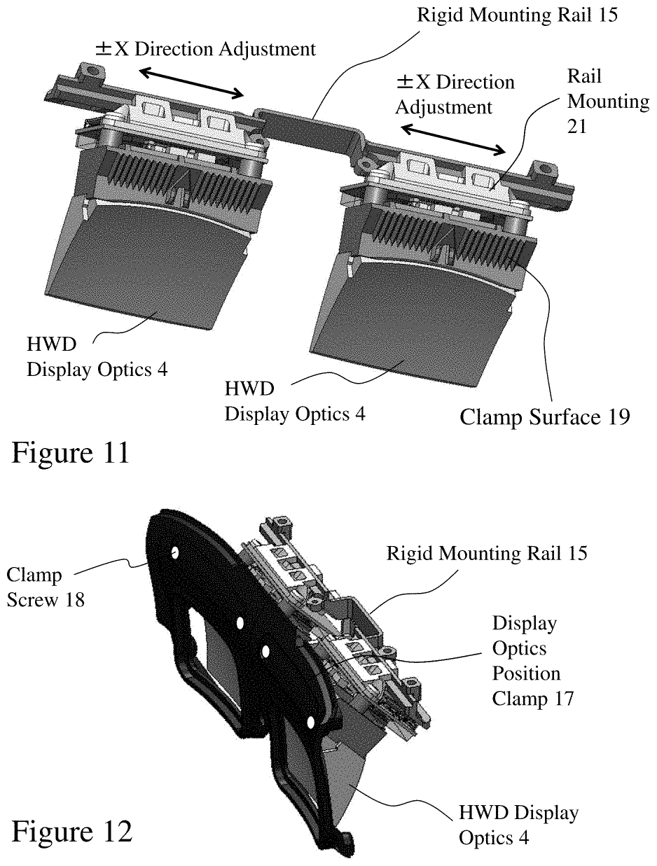

The final step necessary to ensure the HWD Display Optics 4, the wearer's prescription lenses 3, and the wearer's Pupil 5 are all in proper alignment, is to set the horizontal location of the left and right HWD Display Optics 4 according to the wearer's horizontal pupil location. According to an embodiment of the invention as depicted in FIGS. 11 and 12 the HWD Display Optics 4 for the left and right eye are independently mounted on a Rigid Mounting Rail 15, such that they are independently free to slide in the +/−X dimension, or stated differently, left and right. The Ophthalmic Assembly 10, in which the location of trial Lenses 3 having been properly set to the align with the centers of the wearer's left and right pupils, can be used as a reference to set the horizontal location of the HWD Display Optics 4. As depicted the upper portion of each HWD Display Optic 4 includes a Clamp Surface 19.

Finally, according to an embodiment of the invention, the Ophthalmic Assembly 10 and the Electronics Assembly 2 can be simply attached to one another at the hinge points, held firmly in place with rare earth magnets for example, thereby ensuring that the HWD Display Optics 4, the wearer's prescription refractive lenses 3, and their Pupils 5, are in alignment to the required tolerance.

Another advantage of this embodiment of the invention using magnetic coupling between the Ophthalmic Assembly 10 and the Electronics Assembly 2 is that the settings of the Ophthalmic Assembly 10, namely the pupil spacing in the X dimension, Node Bridge Assembly 16 height in the Y dimension, and Node Bridge Assembly 16 depth in the Z dimension, can all be transferred to a second Ophthalmic Assembly 10 in which the trial lenses are replaced with the wearer's refractive lens prescription. In this manner, the Ophthalmic Assembly 10 used by the clinician can be quickly swapped for a less adjustable, more aesthetically pleasing Ophthalmic Assembly 10 that has been customized for the wearer to match the settings of the clinician's Ophthalmic Assembly 10 as it was calibrated for that specific individual. It would be evident to one skilled in the art that the more aesthetically pleasing Ophthalmic Assembly 10 may be one of multiple designs offered by one or more manufacturers as well as by the supplier of the HMD.

According to another aspect of the invention there is presented the use of a Rigid Mounting Rail 15, fabricated from a lightweight rigid material such as for exemplary purposes, titanium, aluminium, silicon carbide, alumina, zirconia, acrylic, polycarbonate, and melamine. Alternatively, fibre reinforced composites may be employed, for example with mineral or carbon fibers. Said left and right HWD Display Optics 4 can move in left/right “X” dimensions on said Rigid Mounting Rail 15, in order to place the HWD display optics assemblies 4 in the correct horizontal location for the individual. The horizontal position of the left and right HWD Display Optics 4 are then held in place by a set screw, compression fit, or other means.

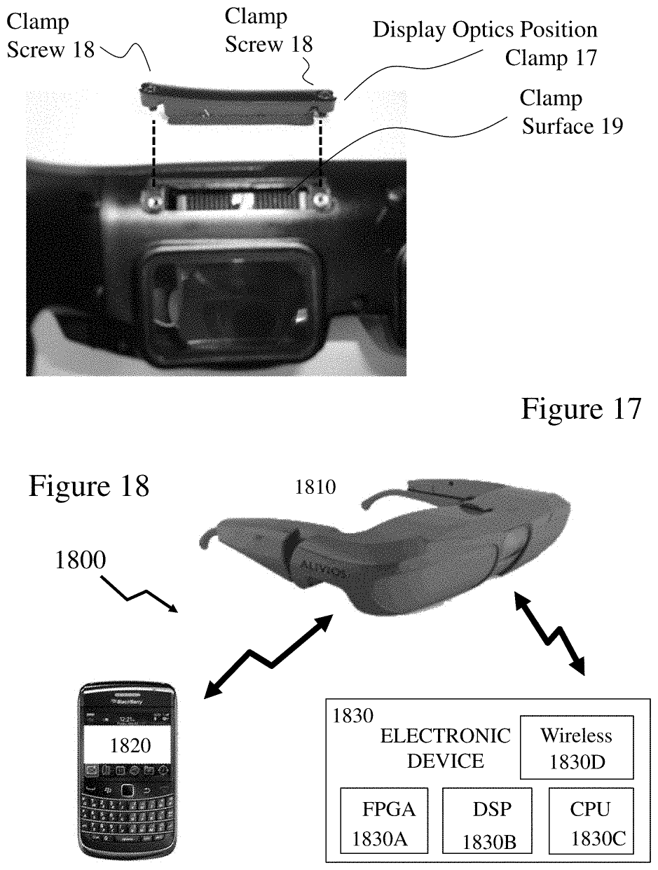

According to an embodiment of the invention as depicted in FIGS. 11 and 12, the HWD Display Optics 4 are held firmly in their respective left/right “X” positions using a Display Optics Position Clamp 17, which mates with a Clamp Surface 19 on each HWD Display Optics assembly 4. In this particular embodiment, the Display Optics Position Clamp 17 is held in place by tightening clamp screws 18, although other methods of seating the Display Optics Position Clamp 17 could be envisioned by one skilled in the art. Another view of this assembly is depicted in FIG. 17 showing the Electronics Assembly 2 with the Display Optics Position Clamp 17 removed, and the Clamp Surface 19 on the HWD display optics assembly in place.

Referring to FIG. 11 depicts an embodiment of the invention wherein a Rigid Mounting Rail 15 is disposed upon which the left and right HWD Display Optics 4 can travel in the “X” direction in order to accommodate different pupil spacings of the user. A Clamp Surface 19 is provided for contact with a Display Optics Position Clamp 17, not shown for clarity, to affix the location of the HWD Display Optics 4 firmly in place on the Rigid Mounting Rail 15 allowing retention post configuration according to an embodiment of the invention.

Now referring to FIG. 12 there is depicted according to an embodiment of the invention the assembly of the Display Optics Position Clamp 17 against the Rigid Mounting Rail 15 which is held in place with two clamp screws 18, firmly mating with the Clamp Surface 19 on the HWD Display Optics 4, thereby preventing said HWD Display Optics 4 from travelling in the left/right “X” dimension once in place. Optionally, Display Optics Position Clamp 17 and Clamp Surface 19 together with the structure of the Rigid Mounting Rail 15 may be modified as would be evident to one skilled in the art to provide alternate adjustment/retaining mechanisms. Alternatively, whilst such an adjustment means may be provided within an Ophthalmic Assembly 10 and employed during fitting the user's final Ophthalmic Assembly 10 it may also comprise a non-adjustable means such that the Ophthalmic Assembly 10 is locked into position when supplied to the user based upon settings established with a trial assembly during a fitting session.

Another view of this assembly is depicted in FIG. 17 wherein the Display Optics Position Clamp 17 has been removed from the Ophthalmic Assembly 10 allowing the Clamp Surface 19 to be seen within allowing the lateral adjustment of HWD Display Optics 4 relative to the wearer's eye(s).

Referring to FIGS. 13 and 14 there are depicted according to an embodiment of the invention, a Node Bridge Assembly 16 which can be adjusted in the vertical up/down “Y” dimension by traversing it with respect to a vertical sliding dovetail channel in the Ophthalmic Assembly 10. Once the correct vertical dimension has been determined for the Node Bridge Assembly 16, such that the HWD Display Optics 4 are properly vertically centered on the wearer's pupils, it can be securely fixed in place using a set screw.

According to an embodiment of the invention the Node Bridge Assembly 16 can be manufactured in a multiplicity of variants, such that the distance of the Ophthalmic Assembly 10 from the wearer's face can be varied controllably by selecting a Node Bridge Assembly 16 with the appropriate in/out “Z” dimension. Optionally, another plurality of Node Bridge Assembly 16 may be manufactured in a different multiplicity of variants allowing the vertical up/down “Y” dimension to be established without traversing the Node Bridge Assembly 16 with respect to a vertical sliding dovetail channel although the final user HWD may employ a sliding Node Bridge Assembly 16 which is set and securely fixed in place whilst the multiplicity of variants provide stability during patient assessment/testing etc. It would be evident to one skilled in the art that preliminary and/or actual measurements for the setting of the Ophthalmic Assembly 10 and Electronic Assembly 2 may be derived from measurements of the user's face alone or in combination with trial and error refinements. Such measurements may be performed with ophthalmic instruments designed for this application as well as with existing ophthalmic instruments which have been modified to increase their functionality. Optionally, physical measurements relating to the Temple Arm 7, Nose Bridge Assembly 16 etc may be obtained from the user directly or indirectly from a mould of the user's facial region for example.

Within another embodiment of the invention the vertical position of each HWD Display Optics 4 relative to the Rigid Mounting Rail 15 may be adjusted by allowing motion of the HWD Display Optics 4 relative to the Rail Mounting 21 and wherein each Display Optics Position Clamp 17 still clamps each HWD Display Optics 4 into position due to the extended vertical dimensions of the Clamp Surface 19.

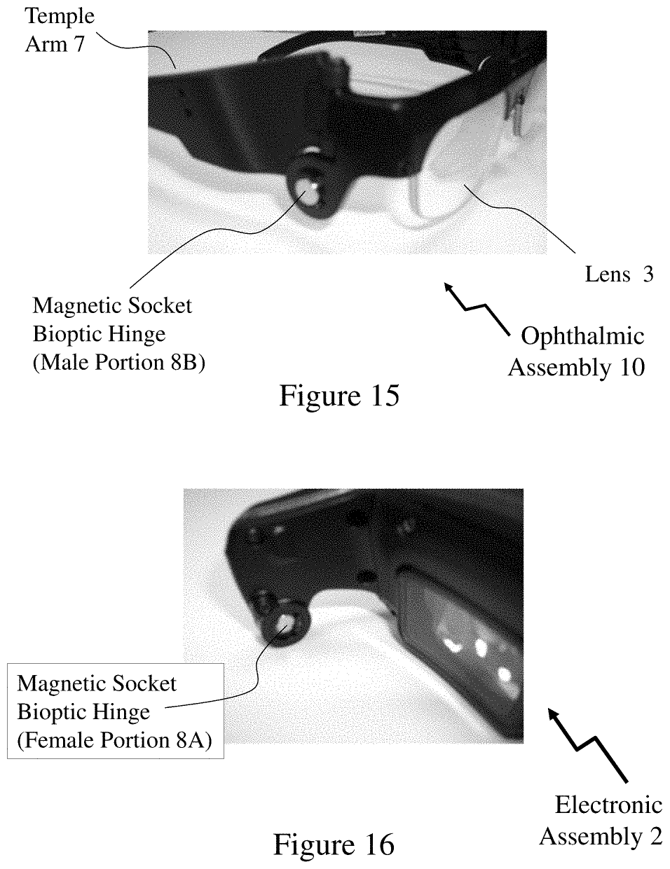

Referring to FIGS. 15 and 16 there are depicted other views of the Ophthalmic Assembly 10 and Electronic Assembly 2 respectively showing in particular the Male Portion 8B and Female Portion 8A respectively of the Magnetic Bioptic Hinge. Within the descriptions supra in respect of FIGS. 1 through 16 respectively focus has been given to the Ophthalmic Assembly 10 and Electronics Assembly 2 from the viewpoint of the optical configuration from display element(s) to the user's eye(s) and hence to aligning the HWD Display Optics 4 within the Electronics Assembly 2 with the Ophthalmic Assembly 10 and these elements to the user's pupils. However, it would be evident that in some embodiments of the invention an alignment process of the Camera 1 with the Electronics Assembly 2 and/or Ophthalmic Assembly 10 may be required as whilst it is anticipated that the Camera 1 will provide wide angle image capture similar to that of the average human the user may have a particular bias in their eye direction relative to a level forward looking head which may be mimicked with the Camera 1. Optionally, the Camera 1 may be on a dynamically adjustable stage allowing the Camera 1 angle to be adjusted according to a sensed parameter such as the user's head tilt. In such embodiments of the invention it may be appropriate to adjust the upper/lower limits of travel according to the preferences of the user. In some embodiments of the invention the Camera 1 may be fixed zoom, adjustable zoom, fixed orientation in X-Y dimensions relative to the user's head, or variable orientation in X-Y dimensions. Optionally, the Camera 1 may be attached to the Ophthalmic Assembly 10 and communicate to the Electronics Assembly 2 via a local wireless protocol, such as Bluetooth for example, or via electrical connections that form additional portions of the Magnetic Bioptic Hinge. For example, contacts may be provided on the Electronics Assembly 2 and Ophthalmic Assembly 10 which support rotation over a predetermined range commensurate with that of the Magnetic Bioptic Hinge.

Within the embodiments of the invention described supra in respect of FIGS. 1 through 7 and 9 through 17 respectively the HWD is described as presenting an image to the user which may be generated from an image captured by a video camera or camera forming part of the HWD, with or without processing for visual impairments relating to the user. According to an embodiment of the invention as depicted in FIGS. 8A and 8B the image presented to the user may be selected as a region of the video or captured image wherein the region selected both in location and size is selected based upon commands provided by the wearer of the HWD.

Now referring to FIG. 18 there is depicted a HWD system 1800 according to an embodiment of the invention wherein a HWD 1810 is coupled to one or more Portable Electronic Devices (PEDs) which provide electronic processing of the image from the camera thereby reducing the requirements on the control and processing electronics within the Electronics Assembly 2. As depicted the PEDs may be a smartphone 1820 or HWD electronics 1830. HWD electronics 1830 comprising an FPGA 1830A for memory and algorithm storage, DSP 1830B for image processing and CPU 1830C wherein image data received from the HWD 1810 via wireless interface 1830D is processed and then re-transmitted to the HWD 1810 for display to the user. Smartphone 1820 provides comparable functionality and may have one or more applications installed to support the graphics processing and control requirements of the HWD 1810.

Accordingly a user wearing HWD 1810 may be provided with enhanced vision through the acquisition of image data; it's processing to address visual defects or visual disorders of the patient, and subsequent presentation to the user through the display and lens assembly. As would be evident from the preceding description such HWDs may be used with or without eyeglasses thereby combining the HWD generated content with the views own visual content received through the optical train comprising HWD lens 420 and eyeglass lens 410 or in some instances may be the sole visual content that the user receives and processes.

As depicted in FIG. 18 the HWD 1810 interfaces to either electronic device 1830 or smartphone 1820. These computing resources may in some instances be replaced by an application specific integrated circuit (ASIC). It would be evident to one skilled in the art that smartphone 1820 and electronic device 1830 may be another portable electronic device (PED) including for example a cellular telephone, portable multimedia player, and portable gaming console. Alternatively the PED may be a dedicated device for the HWD 1810. As depicted within FIG. 18 elements are connected by a wireless link, this may be a wireless link operating for example according to a wireless personal area network (WPAN) or body area network (BAN) standard such as provided by IEEE 802.15 or Bluetooth for example. Optionally, the wireless link may be replaced by or augmented by a wired link which may for example be a HDMI interface although other options are also possible including, but not limited to, RS232, RS485, USB, SPC, I2C, UNI/O, Infiniband, and 1-wire.

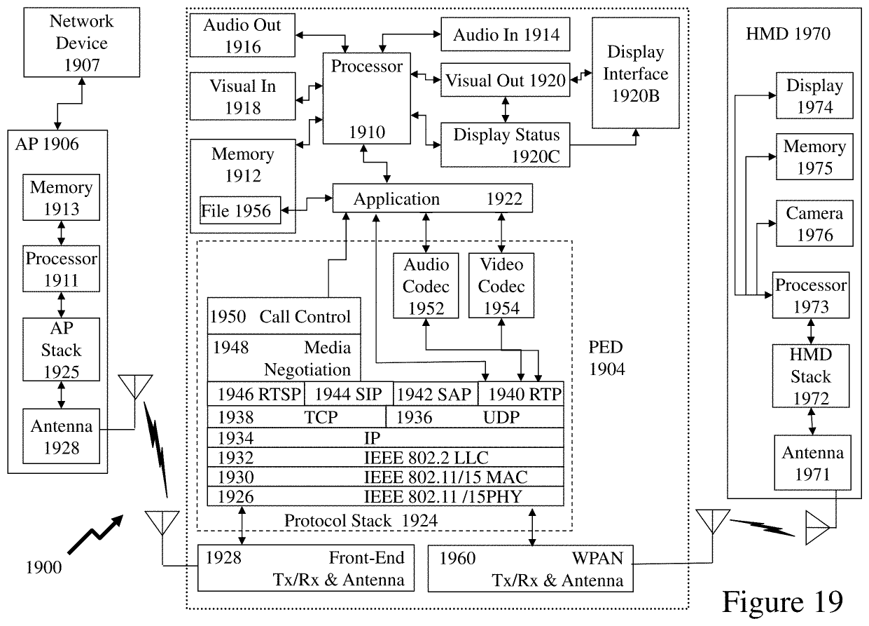

Now referring to FIG. 19 there is depicted a PED 1904 supporting an HWD according to an embodiment of the invention. Also depicted within the PED 1904 is the protocol architecture as part of a simplified functional diagram of a system 1900 that includes a portable electronic device (PED) 1904, such as a smartphone, an access point (AP) 1906, such as first Wi-Fi Access Point 110, and one or more network devices 1907, such as communication servers, streaming media servers, and routers. Network devices 1907 may be coupled to AP 1906 via any combination of networks, wired, wireless and/or optical communication. The PED 1904 includes one or more processors 1910 and a memory 1912 coupled to processor(s) 1910. AP 1906 also includes one or more processors 1911 and a memory 1913 coupled to processor(s) 1911. A non-exhaustive list of examples for any of processors 1910 and 1911 includes a central processing unit (CPU), a digital signal processor (DSP), a reduced instruction set computer (RISC), a complex instruction set computer (CISC) and the like. Furthermore, any of processors 1910 and 1911 may be part of application specific integrated circuits (ASICs), Field Programmable Gate Arrays (FPGAs) or may be a part of application specific standard products (ASSPs). A non-exhaustive list of examples for memories 1912 and 1913 includes any combination of the following semiconductor devices such as registers, latches, ROM, EEPROM, flash memory devices, non-volatile random access memory devices (NVRAM), SDRAM, DRAM, double data rate (DDR) memory devices, SRAM, universal serial bus (USB) removable memory, and the like.

PED 1904 may include an audio input element 1914, for example a microphone, and an audio output element 1916, for example, a speaker, coupled to any of processors 1910. PED 1904 may include a video input element 1918, for example, a video camera, and a visual output element 1920, for example an LCD display, coupled to any of processors 1910. The visual output element 1920 is also coupled to display interface 1920B and display status 1920C. PED 1904 includes one or more applications 1922 that are typically stored in memory 1912 and are executable by any combination of processors 1910. PED 1904 includes a protocol stack 1924 and AP 1906 includes a communication stack 1925. Within system 1900 protocol stack 1924 is shown as IEEE 802.11/15 protocol stack but alternatively may exploit other protocol stacks such as an Internet Engineering Task Force (IETF) multimedia protocol stack for example. Likewise AP stack 1925 exploits a protocol stack but is not expanded for clarity. Elements of protocol stack 1924 and AP stack 1925 may be implemented in any combination of software, firmware and/or hardware. Protocol stack 1924 includes an IEEE 802.11/15-compatible PHY module 1926 that is coupled to one or more Front-End Tx/Rx & Antenna 1928, an IEEE 802.11/15-compatible MAC module 1930 coupled to an IEEE 802.2-compatible LLC module 1932. Protocol stack 1924 includes a network layer IP module 1934, a transport layer User Datagram Protocol (UDP) module 1936 and a transport layer Transmission Control Protocol (TCP) module 1938. Also shown is WPAN Tx/Rx & Antenna 1960, for example supporting IEEE 802.15.

Protocol stack 1924 also includes a session layer Real Time Transport Protocol (RTP) module 1940, a Session Announcement Protocol (SAP) module 1942, a Session Initiation Protocol (SIP) module 1944 and a Real Time Streaming Protocol (RTSP) module 1946. Protocol stack 1924 includes a presentation layer media negotiation module 1948, a call control module 1950, one or more audio codecs 1952 and one or more video codecs 1954. Applications 1922 may be able to create maintain and/or terminate communication sessions with any of devices 1907 by way of AP 1906. Typically, applications 1922 may activate any of the SAP, SIP, RTSP, media negotiation and call control modules for that purpose. Typically, information may propagate from the SAP, SIP, RTSP, media negotiation and call control modules to PHY module 1926 through TCP module 1938, IP module 1934, LLC module 1932 and MAC module 1930.

It would be apparent to one skilled in the art that elements of the PED 1904 may also be implemented within the AP 1906 including but not limited to one or more elements of the protocol stack 1924, including for example an IEEE 802.11-compatible PHY module, an IEEE 802.11-compatible MAC module, and an IEEE 802.2-compatible LLC module 1932. The AP 1906 may additionally include a network layer IP module, a transport layer User Datagram Protocol (UDP) module and a transport layer Transmission Control Protocol (TCP) module as well as a session layer Real Time Transport Protocol (RTP) module, a Session Announcement Protocol (SAP) module, a Session Initiation Protocol (SIP) module and a Real Time Streaming Protocol (RTSP) module, media negotiation module, and a call control module.

Also depicted is HWD 1970 which is coupled to the PED 1904 through WPAN interface between Antenna 1971 and WPAN Tx/Rx & Antenna 1960. Antenna 1971 is connected to HWD Stack 1972 and therein to processor 1973. Processor 1973 is coupled to camera 1976, memory 1975, and display 1974. HWD 1970 being for example system 500 described above in respect of FIG. 5. Accordingly, H W D 1970 may, for example, utilize the processor 1910 within PED 1904 for processing functionality such that a lower power processor 1973 is deployed within HWD 1970 controlling acquisition of image data from camera 1976 and presentation of modified image data to user via display 1974 with instruction sets and some algorithms for example stored within the memory 1975. It would be evident that data relating to the particular individual's visual defects may be stored within memory 1912 of PED 1904 and/or memory 1975 of HWD 1970. This information may be remotely transferred to the PED 1904 and/or HWD 1970 from a remote system such as an optometry system characterising the individual's visual defects via Network Device 1907 and AP 1906.

Accordingly it would be evident to one skilled the art that the HWD with associated PED may accordingly download original software and/or revisions for a variety of functions including diagnostics, display image generation, and image processing algorithms as well as revised ophthalmic data relating to the individual's eye or eyes. Accordingly, it is possible to conceive of a single generic HWD being manufactured that is then configured to the individual through software and patient ophthalmic data. Optionally, the elements of the PED required for network interfacing via a wireless network (where implemented), HWD interfacing through a WPAN protocol, processor, etc may be implemented in a discrete standalone PED as opposed to exploiting a consumer PED. A PED such as described in respect of FIG. 19 allows the user to adapt the algorithms employed through selection from internal memory, to define a Region of Interest (ROI), and otherwise control the operation of the HWD through a touchscreen, touchpad, or keypad interface for example.

It would be evident to one skilled in the art that in some circumstances the user may elect to load a different image processing algorithm and/or HWD application as opposed to those provided with the HWD. For example, a third party vendor may offer an algorithm not offered by the HWD vendor or the HWD vendor may approve third party vendors to develop algorithms addressing particular requirements. Optionally the HWD can also present visual content to the user which has been sourced from an electronic device, such as a television, computer display, multimedia player, gaming console, personal video recorder (PVR), or cable network set-top box for example. This electronic content may be transmitted wirelessly for example to the HWD directly or via a PED to which the HWD is interfaced. Alternatively the electronic content may be sourced through a wired interface such as USB, I2C, RS485, etc as discussed above.

Accordingly a user may employ a software control application on their PED 1904 to dynamically adjust the region of interest (ROI) and/or magnification of the image captured by the camera within their HWD which is displayed upon the display(s) of their HWD. Optionally, such a ROI/magnification selection may also be applied in conjunction with other visual processing effects which address the visual impairment(s) of the user such as re-mapping the image to avoid damaged portions of the retina, re-colour mapping to correct for colour blindness, dithering edges to increase visual contrast through triggering processes within the visual cortex. Within other embodiments of the invention the software control application on their PED 1904 may also control functions in respect of the Camera 1 such as zoom, pan, tilt etc allowing increased visual function for a user with restricted range of motion of their eyes and/or neck.

Within the preceding descriptions of embodiments of the invention with respect to FIGS. 1 through 19 that Ophthalmic Assembly 10 may be implemented using design concepts similar to those in spectacles (eyeglasses) as well as exploiting other approaches to

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. A device comprising:
(i) an ophthalmic assembly to be worn by a user comprising:
a frame to fit along a left side and a right side of the user's head;
a first portion of a first bioptic hinge disposed on a first portion of the frame which fits along the left side of the user's head for coupling to a second portion of the first bioptic hinge on an electronics assembly allowing demountable coupling of the electronics assembly to and from the ophthalmic assembly;
a first portion of a second bioptic hinge disposed on a second portion of the frame which fits along the right side of the user's head for coupling to a second portion of the second bioptic hinge on the electronics assembly allowing demountable coupling of the electronics assembly to and from the ophthalmic assembly; and
(ii) the electronic assembly comprising
an electronic processor for:
receiving data from at least one of a remote source and a camera; and
generating image data in real time in dependence upon the at least one of the remote source and the camera for display to the user via a pair of near-to-eye displays also forming part of the electronic assembly;
the second portion of the first bioptic hinge disposed on a first portion of the electronic assembly for coupling to the first portion of the first bioptic hinge of the ophthalmic assembly;
the second portion of the second bioptic hinge mounting disposed on a second portion of the electronic assembly for coupling to the first portion of the second bioptic hinge of the ophthalmic assembly; wherein
the electronics assembly is configured to position the pair of near-to-eye displays relative to the user's eyes when mounted to the ophthalmic assembly;
the user wears the ophthalmic assembly either with or without the electronic assembly attached;
the first portion of the first bioptic hinge mounting and second portion of the first bioptic hinge when coupled to each other together with the first portion of the second bioptic hinge and the second portion of the second bioptic hinge when coupled to each other each provide a pivot point for the electronics assembly allowing it to be pivoted vertically relative to the ophthalmic assembly.

2. The device according to claim 1, wherein
the electronics assembly pivots as a single unit relative to the ophthalmic assembly.

3. The device according to claim 1, wherein
the electronics assembly is only attached to the ophthalmic assembly by the first bioptic hinge and the second bioptic hinge.

4. The device according to claim 1, wherein
the first bioptic hinge is positioned proximate the left temple of the user's head;
the second bioptic hinge is positioned proximate the right temple of the user's head;
the electronics assembly when pivoted vertically relative to the ophthalmic assembly allows the user to still view content displayed on the near-to-eye display over a predetermined portion of the range of pivotal vertical motion of the electronics assembly relative to the ophthalmic assembly.

5. A device comprising:
(i) an assembly to be worn by a user comprising:
a load bearing frame to fit around the user's head including along a left side if the user's head and a right side of the user's head;
a first portion of a first bioptic hinge disposed on a first portion of the frame which fits along the left side of the user's head for coupling to a second portion of the first bioptic hinge on an electronics assembly allowing demountable coupling of the electronics assembly to and from the ophthalmic assembly;
a first portion of a second bioptic hinge disposed on a second portion of the frame which fits along the right side of the user's head for coupling to a second portion of the second bioptic hinge on the electronics assembly allowing demountable coupling of the electronics assembly to and from the ophthalmic assembly; and
(ii) an electronic assembly comprising:
an electronic processor for receiving data from at least one of a remote source and a camera and generating image data in real time in dependence upon the at least one of the remote source and the camera for display to the user via a pair of near-to-eye displays also forming part of the electronic assembly;
the second portion of the first bioptic hinge disposed on a first portion of the electronic assembly for coupling to the first portion of the first bioptic hinge of the ophthalmic assembly;
the second portion of the second bioptic hinge mounting disposed on a second portion of the electronic assembly for coupling to the first portion of the second bioptic hinge of the ophthalmic assembly; wherein
the electronics assembly is configured to position the pair of near-to-eye displays relative to the user's eyes;
a first near-to-eye display of the pair of near-to-eye displays within the electronic assembly is laterally aligned to the user's left eye independent of both the position of a second near-to-eye display of the pair of near-to-eye displays and the user's right eye;
the second near-to-eye display of the pair of near-to-eye displays within the electronic assembly is laterally aligned to the user's right eye independent of both the position of the first near-to-eye display of the pair of near-to-eye displays and the user's left eye; and
each of the first near-to-eye display of the pair of near-to-eye displays and the second near-to-eye display of the pair of near-to-eye displays are laterally positioned independent of the user's nose.

6. The device according to claim 5, wherein
each of the first near-to-eye display of the pair of near-to-eye displays and the second near-to-eye display of the pair of near-to-eye displays are laterally positioned independent of any nose bridge forming part of the ophthalmic assembly.

7. The device according to claim 5, wherein
the first bioptic hinge and the second bioptic hinge provide demountable couplings for the electronics assembly allowing it to be mounted and demounted to the ophthalmic assembly; and
the first bioptic hinge and the second bioptic hinge provide pivot points for the electronics assembly allowing it to be pivoted vertically relative to the ophthalmic assembly when they are coupled together.

8. The device according to claim 5, wherein
the electronics assembly pivots as a single unit relative to the ophthalmic assembly.

9. The device according to claim 5, wherein
the electronics assembly is only attached to the ophthalmic assembly by the first bioptic hinge and the second bioptic hinge.

10. A method comprising:
providing a first assembly to be worn by a user comprising:
a first frame to fit along a left side and a right side of the user's head;
a first portion of a first bioptic hinge disposed on a first portion of the frame which fits along the left side of the user's head for coupling to a second portion of the first bioptic hinge on an electronics assembly allowing demountable coupling of the electronics assembly to and from the first assembly;
a first portion of a second bioptic hinge disposed on a second portion of the frame which fits along the right side of the user's head for coupling to a second portion of the second bioptic hinge on the electronics assembly allowing demountable coupling of the electronics assembly to and from the first assembly;
providing a second assembly to be worn by the user comprising:
a second frame to fit along the left side and the right side of the user's head;
a first portion of another first bioptic hinge disposed on a first portion of the second frame which fits along the left side of the user's head for coupling to the second portion of the first bioptic hinge on the electronics assembly allowing demountable coupling of the electronics assembly to and from the second assembly; and
a first portion of another second bioptic hinge disposed on a second portion of the second frame which fits along the right side of the user's head for coupling to the second portion of the first bioptic hinge on the electronics assembly allowing demountable coupling of the electronics assembly to and from the second assembly;
providing an electronic assembly comprising:
an electronic processor for receiving data from at least one of a remote source and a camera and generating image data in real time in dependence upon the at least one of the remote source and the camera for display to the user via a pair of near-to-eye displays also forming part of the electronic assembly;
the second portion of the first bioptic hinge disposed on a first portion of the electronic assembly for coupling to either the first portion of the first bioptic hinge of the first assembly or the first portion of the another first bioptic hinge of the second assembly;
the second portion of the second bioptic hinge mounting disposed on a second portion of the electronic assembly for coupling to either the first portion of the second bioptic hinge of the first assembly or the first portion of the another second bioptic hinge of the second assembly; and
performing an adjustment process with the user wearing the second assembly to:
align and fix into position a first near-to-eye display of the pair of near-to-eye displays within the electronic assembly with respect to the user's left eye independent of both the position of a second near-to-eye display of the pair of near-to-eye displays and the user's right eye; and
align and fix into position the second near-to-eye display of the pair of near-to-eye displays within the electronic assembly with respect to the user's right eye independent of both the position of the first near-to-eye display of the pair of near-to-eye displays and the user's left eye; wherein
the electronics assembly is configured to position the pair of near-to-eye displays relative to the user's eyes when mounted to the first assembly;
the user wears the first assembly either with or without the electronic assembly attached;
the user wears the second assembly either with or without the electronic assembly attached;
the first assembly can be interchangeably attached to the electronic assembly with the second assembly; and
the second assembly further comprises a mount for mounting a plano trial lens with a predetermined pattern to the second assembly to establish alignment of a center of the plano trial lens with a pupil of the user's eye during the adjustment process.

11. The method according to claim 10, wherein
the first assembly comprises a first nose bridge of a plurality of nose bridges;
the second assembly further comprises a second nose bridge of the plurality of nose bridges;
each of the first assembly and second assembly are employed individually with the electronics assembly;
each nose bridge of the plurality of nose bridges defines when individually attached to either the first assembly or the second assembly at least one of:
a distance between the user's eye and the pair of near-to-eye displays;
a vertical position of a front portion of the first assembly when attached to the first assembly and when worn by the user relative to an eyeline of the user; and
a vertical position of a front portion of the second assembly when attached to the second assembly and worn by the user relative to the eyeline of the user.

12. The method according to claim 10, wherein
the first assembly comprises a first nose bridge of a plurality of nose bridges;
the second assembly further comprises a second nose bridge of the plurality of nose bridges;
each of the first assembly and second assembly are employed individually with the electronics assembly;
each nose bridge of the plurality of nose bridges defines when individually attached to either the first assembly or the second assembly a minimum distance between the user's eye and the pair of near-to-eye displays when the electronics assembly and a vertical position of the pair of near-to-eye displays relative to an eyeline of the user.
the nose bridge is one of a plurality of nose bridges where each nose bridge when individually attached to the assembly independent of any other nose bridge of the plurality of nose bridges defines a different minimum distance.

13. A device comprising:
(i) an assembly to be worn by a user comprising:
a frame to fit along a left side and a right side of the user's head;
a first portion of a first bioptic hinge disposed on a first portion of the frame which fits along the left side of the user's head for coupling to a second portion of the first bioptic hinge on an electronics assembly allowing demountable coupling of the electronics assembly to and from the ophthalmic assembly;
a first portion of a second bioptic hinge disposed on a second portion of the frame which fits along the right side of the user's head for coupling to a second portion of the second bioptic hinge on the electronics assembly allowing demountable coupling of the electronics assembly to and from the ophthalmic assembly; and
(ii) an electronic assembly comprising:
an electronic processor for receiving data from at least one of a remote source and a camera and generating image data in real time in dependence upon the at least one of the remote source and the camera for display to the user via a pair of near-to-eye displays also forming part of the electronic assembly;
the second portion of the first bioptic hinge disposed on a first portion of the electronic assembly for coupling to the first portion of the first bioptic hinge of the ophthalmic assembly;
the second portion of the second bioptic hinge mounting disposed on a second portion of the electronic assembly for coupling to the first portion of the second bioptic hinge of the ophthalmic assembly;
a mounting rail comprising a clamp surface having first features;
a first display assembly having a first portion comprising second features for mating to a first subset of the first features and a second portion comprising a first near-to-eye display of the pair of near-to-eye displays;
a second display assembly having a first portion comprising further second features for mating to a second subset of the first features and a second portion comprising a second near-to-eye display of the pair of near-to-eye displays; wherein
the electronics assembly is configured to position the pair of near-to-eye displays relative to the user's eyes when mounted to the assembly;
the first display assembly and thereby the first near-to-eye display of the pair of near-to-eye displays is aligned to the user's left eye independent of both the position of the second near-to-eye display of the pair of near-to-eye displays and the user's right eye and fixed into position with the first plurality of second features engaged against a first predetermined portion of the plurality of first features;
the second display assembly and thereby the second near-to-eye display of the pair of near-to-eye displays is aligned to the user's right eye independent of both the position of the first near-to-eye display of the pair of near-to-eye displays and the user's right eye and fixed into position with the second plurality of second features engaged against a second predetermined portion of the plurality of first features; and
each of the first near-to-eye display of the pair of near-to-eye displays and the second near-to-eye display of the pair of near-to-eye displays are positioned independent of any symmetry relative to both the user's nose and the user's head.

14. The device according to claim 13, wherein
the assembly further comprises a nose bridge a plurality of nose bridges; and
each nose bridge of the plurality of nose bridges defines when individually attached to the assembly independent of any other bridge of the plurality of bridges establishes at least one of:
a distance between the user's eye and the near-to-eye display when the electronics assembly is attached to the assembly; and
a vertical position of a front portion of the frame assembly at a front of the user's head when worn by the user relative to an eyeline of the user.
